(19) United States
(12) Patent Application Publication
(10) Pub. No.: US 2025/0387614 A1
(43) Pub. Date: Dec. 25, 2025

(54) SYSTEMS, APPARATUSES, DEVICES AND METHODS OF LEAK DETECTION FOR PNEUMATIC CARDIAC ASSIST DEVICES

(71) Applicant: NuPulseCV, Inc., Raleigh, NC (US)

(72) Inventors: Nagarjuna Reddy CHINTA, Apex, NC (US); Guruprasad Anapathur GIRIDHARAN, Louisville, KY (US); Douglas Edward IVERS, Cary, NC (US)

(21) Appl. No.: 19/315,028

(22) Filed: Aug. 29, 2025

Related U.S. Application Data
(63) Continuation of application No. PCT/US2025/025869, filed on Apr. 22, 2025.
(60) Provisional application No. 63/638,772, filed on Apr. 25, 2024.

(51) Int. Cl. A61M 60/497; A61M 60/554

(52) U.S. Cl. CPC A61M 60/497; A61M 60/554; A61M 2205/15

(57) ABSTRACT

A pneumatic cardiac assist device (PCAD) leak detection apparatus and method is described. The embodiments offer efficient and effective detection of leaks in PCADs, including but not limited to counterpulsation devices. A leak is detected based on measured pressures and temperatures at a first time and at a second time and further while accounting for a change in PCAD system pressure that is attributable to the change in temperature over the period of time. The PCAD system may be controlled by the leak detection.

### RELATED APPLICATIONS

The present application is a continuation of International Patent Application No. PCT/US2025/025869, filed Apr. 22, 2025, entitled “Systems, Apparatuses, Devices and Methods of Leak Detection for Pneumatic Cardiac Assist Devices,” which claims benefit of and priority to U.S. Provisional Application No. 63/638,772, filed Apr. 25, 2024, entitled, “Leak Detection Apparatus and Method for Pneumatic Cardiac Assist Devices”, the entire disclosure of each of which is herein incorporated by reference for any and all purposes.

The following disclosures are herein incorporated by reference in their entireties for any and all purposes for the invention(s) and embodiments of this subject application:
U.S. Pat. Nos. 7,892,162, 8,066,628, 8,323,174, 8,326,421, 10,137,230, and 12,156,978,
U.S. application nos.:
   Ser. No. 17/944,130, titled “Intra-aortic balloon pump assembly with pressure sensor” and published as US2024/0082565 on Mar. 14, 2024;
   Ser. No. 17/944,127, titled “Intra-aortic balloon pump assembly” and published as US2024/0082564 on Mar. 14, 2024;
   Ser. No. 17/944,125, titled “Blood pump support apparatus and method for a blood pump assembly” and published as US2024/0082563 on Mar. 14, 2024; and
   Ser. No. 17/878,632, titled “Drive unit for intravascular circulatory support systems” and published as US2023/0069771 on Mar. 2, 2023.

### FIELD

One or more embodiments herein relate to apparatus and methods for detecting leaks from pneumatic cardiac assist devices.

### BACKGROUND

The disclosure relates generally to medical devices, and more particularly to systems and methods related to the operation of pneumatic cardiac assist devices (CADs). About 5.7 million adults in the United States have heart failure, according to the U.S. Centers for Disease Control and Prevention website. Each year, about 100,000 people nationally are diagnosed with advanced heart failure and require some sort of mechanical support, e.g., via a CAD. A pneumatic CAD (PCAD) is a CAD that operates pneumatically (e.g., using a gas such as air). In some instances, a PCAD may perform ventricular assistance, counterpulsation therapy or copulsation therapy. In general terms, counterpulsation is a ventricular support technique that assists circulation and decreases the work of the heart by increasing aortic blood pressure during diastole (i.e., diastolic aortic pressure) relative to unassisted diastolic aortic blood pressure (i.e., without counterpulsation) and by decreasing aortic blood pressure during systole (i.e., systolic aortic blood pressure) relative to unassisted systolic aortic blood pressure (i.e., without counterpulsation). For its part, copulsation is a ventricular support technique that increases systolic aortic blood pressure relative to unassisted systolic aortic blood pressure.

PCADs may include intra-aortic balloon pumps, patch devices, cuff devices, and other pump devices anastomosed to a large blood vessel. Such devices may be utilized to perform counterpulsation. For example, the intra-aortic balloon pump or IABP may be positioned inside the aorta, typically in the proximal descending aorta. The balloon pump (typically 25-50 milliliters in capacity) may be inflated and deflated in synchrony with the contraction of the left ventricle, thereby performing counterpulsation therapy. The IABP may be inflated during diastole, thereby increasing diastolic aortic blood pressure relative to the unassisted diastolic aortic blood pressure and driving blood in the ascending aorta and aortic arch into the coronary arteries to supply oxygen to the heart muscle. The IABP may be deflated during systole, as the left ventricle contracts, thereby decreasing systolic aortic blood pressure relative to unassisted systolic aortic blood pressure and decreasing the afterload (i.e., the effort required of the heart to push blood into the aorta). As used here, the term “inflated” includes partially inflated and “deflated” includes partially deflated.

Similarly, patch devices may be implanted on the descending thoracic aorta. In particular, a longitudinal incision on the descending thoracic aorta may be performed and the patch device may be sutured to the lateral aspect of the descending thoracic aorta. The patch device may include an elongated inflatable valve-less polyurethane patch powered by a drive unit (e.g., an external compressor). The patch may be inflated and deflated in time with the contraction of the left ventricle, thereby performing counterpulsation therapy, similar to the manner described above with respect to the balloon pump. An example of a patch device is the Kantrowitz CardioVad patch device.

Similarly, cuff devices may be wrapped around the ascending aorta and may have a membrane (e.g., a balloon) that is positioned against the vessel's external wall. The positive and negative pressure of the membrane facilitates counterpulsation. An example of a cuff device is the Sunshine C-Pulse T heart assist system.

Blood pump devices may be implanted into a pocket below the petoralis muscle on the anterior chest and attached to a vascular graft anastomosed to the subclavian artery. The blood pump device may include a reservoir that fills or partially fills with blood during systole (thereby reducing systolic aortic blood pressure relative to unassisted systolic aortic blood pressure) and empties during diastole (thereby increasing diastolic aortic blood pressure relative to unassisted diastolic aortic blood pressure). An example of such a blood pump device is the Symphony Counter Pulsation Device (CPD). Other blood pump devices exist including so-called para-aortic blood pumps and disposable blood pumps for life support during cardiac surgical procedures (e.g., the PulseCath iVAC or PUCA pump). Both operate similarly to the Symphony CPD. Para-aortic blood pumps are anastomosed to the descending aorta and the PulseCath iVAC or PUCA pumps consist of a catheter containing a two-way value connected to a pump that operates to aspirate blood from the left ventricle in systole (decreasing systolic aortic blood pressure relative to unassisted systolic aortic blood pressure) and push the blood back into the aorta during diastole (increasing diastolic aortic blood pressure relative to unassisted diastolic aortic blood pressure).

Notably, PCADs may be utilized to perform copulsation, if implanted in the ventricle and configured to increase aortic blood pressure (relative to the unassisted aortic blood pressure) during systole.

PCADs may be a component in a PCAD system that also includes a driveline and a drive unit. The drive unit may be configured to generate gas pressure and gas flow to operate the PCAD, and the driveline or drivelines may be configured to shuttle the gas from the pneumatic drive unit to and from the PCAD.

In operation, a leak in a PCAD system can prevent the PCAD from performing effective therapy for the patient or may be harmful or fatal to a patient. For example, a leak anywhere in the PCAD system may affect the ability of the PCAD to inflate and/or deflate and/or create positive/negative pressure and/or otherwise pump, thereby leading to ineffective cardiac therapy. Similarly, a leak in the driveline or PCAD may result in gas being communicated into the vasculature, which could be harmful, poisonous and/or fatal to the patient. Accordingly, a need exists for detecting a leak in the PCAD system and controlling the drive unit in response to such a detection.

### SUMMARY OF AT LEAST SOME EMBODIMENTS OF THE DISCLOSURE

In some embodiments of the present disclosure, an apparatus includes a leak detection module operative to determine if there is a leak in a pneumatic cardiac assist device (PCAD) system over a period of time defined by a first time and a second time. The determination is based on a first PCAD system pressure at the first time (P1), a first PCAD system temperature at the first time (T1), a second PCAD system pressure at the second time (P2), and a second PCAD system temperature at the second time (T2), while accounting for a change in PCAD system pressure over the period of time attributable to a difference between T2 and T1. P1, T1, P2, and T2 are associated with at least one component of the PCAD system. The apparatus also includes a leak control module that is operative to control the PCAD system in response to a determination that there is a leak in the PCAD system by the leak detection module.

Such embodiments may include one and/or another of (and in some embodiments, if not mutually exclusive, a plurality of, in some embodiments, a majority of, in some embodiments, substantially all of, and in some embodiments, all of) the following features, functionality steps, structure and clarifications:
a pressure sensor operative to determine P1 and P2;
a temperature sensor operative to determine T1 and T2;
the PCAD system includes a pneumatic drive unit, a driveline, and the PCAD;
   the pneumatic drive unit is operative to generate gas pressure and gas flow to operate the PCAD;
   the driveline is operative to shuttle the gas from the pneumatic drive unit to the PCAD;
   the PCAD is operative to provide therapeutic care to a patient;
   the PCAD is one of: an intra-aortic balloon, a patch device, a cuff device, and a pump device;
   the pneumatic drive unit includes a manifold that is operative to communicate gas to the driveline;
      P1 and P2 are the pressure of gas within the manifold at the first and second times, respectively;
      T1 and T2 are the temperature of gas within the manifold at the first and second times, respectively;
      the gas is air;
the leak detection module determines a leak using the Pressure-Temperature Gas Law; the leak detection module is operative to account for the change in PCAD system pressure attributable to the difference between T1 and T2 by determining a temperature dependent pressure change at the second time based on the difference between T1 and T2;
   the temperature dependent pressure change represents an expected difference in the PCAD system over the period of time given the change in T1 and T2;
   the temperature dependent pressure change is defined as the product of (a) P1 on an absolute pressure scale (P1, ABS) over T1 on an absolute temperature scale (T1, ABS) and (b) the difference in T2 and T1, and T1 and T2 are expressed on the same temperature scale, said same temperature scale being one of the absolute temperature scale and a complementary temperature scale;
   the leak detection module is operative to convert P1 into P1, ABS;
   the leak detection module is operative to convert T1 into T1, ABS;
   the leak detection module is further operative to determine a temperature-compensated pressure at the second time based on P2 and the temperature dependent pressure change;
      the temperature-compensated pressure is defined as P2 reduced by the temperature dependent pressure change;
      the leak detection module is further operative to determine that there is a leak in the PCAD system when the temperature compensated pressure is one of: less than P1 and more than insubstantially less than P1;
the leak detection module is operative to account for the change in PCAD system pressure attributable to the difference between T1 and T2 by determining an expected PCAD system pressure at the second time based on the difference between T1 and T2;
   the expected PCAD system pressure at the second time is defined as the sum of P1 and the product of (a) P1 on an absolute pressure scale (P1, ABS) over T1 on an absolute temperature scale (T1, ABS) and (b) the difference in T2 and T1, P1 is expressed on one of the absolute pressure scale and a complementary pressure scale, and T1 and T2 are expressed on the same temperature scale, said same temperature scale being one of the absolute temperature scale and a complementary temperature scale;
   the leak detection module is operative to convert P1 into P1, ABS;
   the leak detection module is operative to convert T1 into T1, ABS;
   the leak detection module is operative to determine that there is a leak in the PCAD system when P2 is one of: less than the expected PCAD system pressure and more than insubstantially less than the expected PCAD system pressure; and
the leak control module is operative to cease actuation of the PCAD system upon a determination that there is a leak in the PCAD system; and
the PCAD system;
   the PCAD system is configured to operate in both a closed mode and in an open mode, when the PCAD system is configured to operate in a closed mode, air is not permitted to enter or escape the system, when the PCAD system is configured to operate in an open mode, air is permitted to enter and escape the system; and each of the first time and the second time are times during which the PCAD system is operating in closed mode.

In some embodiments of the present disclosure, a method includes determining there is a leak in a pneumatic cardiac assist device (PCAD) system over a period of time defined by a first time and a second time. The determination is based on a first PCAD system pressure at the first time (P1), a first PCAD system temperature at the first time (T1), a second PCAD system pressure at the second time (P2), and a second PCAD system temperature at the second time (T2) while accounting for a change in PCAD system pressure over the period of time attributable to a difference between T2 and T1, P1, T1, P2, and T2 are associated with at least one component of the PCAD system. The method also includes controlling the PCAD system in response to the determination that there is a leak in the PCAD system.

Such embodiments may include one and/or another of (and in some embodiments, if not mutually exclusive, a plurality of) the following features, functionality, steps, structure, and clarifications:
the PCAD system includes a pneumatic drive unit, a driveline, and the PCAD;
   the pneumatic drive unit is operative to generate gas pressure and gas flow to operate the PCAD;
      the driveline is operative to shuttle the gas from the pneumatic drive unit to the PCAD;
      the gas is air:
   the PCAD is operative to provide therapeutic care to a patient;
   the PCAD is one of: an intra-aortic balloon, a patch device, and a cuff device;
the determination is based on the Pressure-Temperature Gas Law;
accounting for the change in PCAD system pressure attributable to the difference between T1 and T2 includes determining a temperature dependent pressure change at the second time based on the difference between T1 and T2, wherein the temperature dependent pressure change represents an expected difference in the PCAD system over the period of time given the change in T1 and T2.
   the temperature dependent pressure change is defined as the product of (a) P1 on an absolute pressure scale (P1, ABS) over T1 on an absolute temperature scale (T1, ABS) and (b) the difference in T2 and T1, and T1 and T2 are expressed on the same temperature scale, said same temperature scale being one of the absolute temperature scale and a complementary temperature scale.
   determining a temperature compensated pressure at the second time based on P2 and the temperature dependent pressure change, wherein the temperature-compensated pressure is defined as P2 reduced by the temperature dependent pressure change.
      determining that the temperature compensated pressure is one of: less than P1 and more than insubstantially less than P1.
accounting for the change in PCAD system pressure attributable to the difference between T1 and T2 includes determining an expected PCAD system pressure at the second time associated with the difference between T1 and T2.
   the expected PCAD system pressure at the second time is defined as the sum of P1 and the product of (a) P1 on an absolute pressure scale (P1, ABS) over T1 on an absolute temperature scale (T1, ABS) and (b) the difference in T2 and T1, P1 is expressed on one of the absolute pressure sale and a complementary pressure scale, and T1 and T2 are expressed on the same temperature scale, said same temperature scale being one of the absolute temperature scale and a complementary temperature scale.
   determining that P2 is one of less than the expected PCAD system pressure and more than insubstantially less than the expected PCAD system pressure.
ceasing actuation of the PCAD system.

These and other embodiments of the inventions disclosed herein will be made even more clear by reference to the following detailed description and drawings, a brief description of which is provided immediately below.

### BRIEF DESCRIPTION OF THE DRAWINGS

The accompanying drawings illustrate several embodiments and, together with the description, serve to explain the disclosed principles. One skilled in the relevant art will understand, however, that embodiments can be practiced without all of the specific details of the illustrated examples. Likewise, one skilled in the relevant art will also understand that the technology may include well-known structures or functions not specifically illustrated to avoid unnecessarily obscuring the relevant descriptions of the various examples. In the drawings:

FIG. 1 illustrates an exemplary PCAD system with an intra-aortic balloon pump implanted within a patient's vasculature and configured in accordance with embodiments of the present technology.

FIGS. 2A, 2B, 2C, 2D, 2E, and 2F illustrate an exemplary drive unit capable of use in the exemplary PCAD system of FIG. 1 in accordance with some embodiments of the present technology.

FIG. 3 Illustrates a functional block diagram of an exemplary drive unit including a drive unit control module in accordance with some embodiments of the present technology.

FIG. 4 illustrates a functional block diagram of an exemplary leak detection apparatus in communication with an exemplary drive unit in accordance with some embodiments of the present technology.

FIG. 5 illustrate another functional block diagram of an exemplary leak detection apparatus in communication with an exemplary drive unit in accordance with some embodiments of the present technology.

FIGS. 6A, 6B, 6C, 6D, and 6E illustrate an exemplary manifold in accordance with some embodiments of the present technology.

FIG. 7 illustrates a flowchart including an exemplary method for detecting leaks within a PCAD in accordance with some embodiments of the present technology.

### DETAILED DESCRIPTION

While the disclosed technology may be particularly useful for counterpulsation and copulsation, it may have application to other therapeutic modalities. Several embodiments are discussed below in more detail in reference to the figures. Other embodiments in addition to those described herein are within the scope of the present technology. Moreover, a person of ordinary skill in the art will understand that embodiments of the present technology may have configurations, components, and/or procedures in addition to those shown or described herein and that these and other embodiments may be implemented without several of the configurations, components, and/or procedures shown or described herein without deviating from the present technology. Reference throughout this description to “one embodiment,” “an embodiment,” “one or more embodiments,” an “nth embodiment,” or “some embodiments” means that a particular feature, support structure or characteristic described in connection with the embodiment is included in at least one embodiment of the present disclosure. Thus, use of such terminology is not necessarily referring to the same embodiment. For example, it is expressly contemplated that the features described herein may be combined in any suitable manner in one or more embodiments.

### EXEMPLARY PCAD SYSTEM COMPONENTS

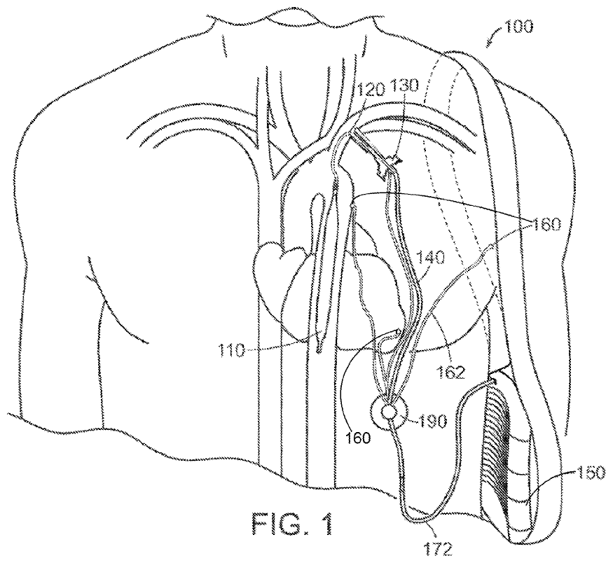

PCAD system may include a PCAD, a driveline, and a drive unit. FIG. 1 illustrates an exemplary PCAD system 100 with intra-aortic balloon pump PCAD 110 implanted within a patient's vasculature. Although the PCAD in FIG. 1 is depicted as an IABP 110, PCAD may alternatively be a patch device (not depicted), a cuff device (not depicted) or other blood pump (not depicted) implantable in or on an aorta of a patient. Exemplary PCAD system 100 may further include a first driveline 120 (also referred to as an “internal driveline”), an arterial interface device or stopper device 130, a second driveline 140, a skin or patient interface device 190, pneumatic driveline/tube 172, a drive unit 150, and sensors 160. In some embodiments, the PCAD system 100 may have certain features generally similar to those described in U.S. patent application Ser. No. 16/876,110, the disclosure of which is incorporated herein by reference in its entirety. When PCAD 110 is implanted, PCAD system 100 may provide counterpulsation therapy to a patient. One of skill in the art will appreciate that PCAD system 100 can be configured to provide copulsation or other therapies to the patient.

Balloon pump 110 may be an expandable member, a balloon or other element that may change size and/or shape in response to being filled with a gas. For example, in some embodiments the balloon pump 110 is a balloon composed of a biocompatible, non-thrombogenic elastomeric material (e.g., Biospan®-S). Balloon pump 110 may also be made of other suitable materials. Balloon pump 110 may be transitioned between at least a first state in which it is generally deflated and a second state in which it is generally inflated. The balloon pump 110 may have a first volume when in the first (e.g., deflated) state and a second volume that is greater than the first volume when in the second (e.g., inflated) state. Accordingly, the balloon pump 110 may provide counterpulsation therapy by repeatedly transitioning between the first state and the second state. To transition the balloon pump 110 between the first state and the second state, the drive unit 150 may direct a gas (e.g., air) into an internal volume of the balloon pump 110 via the first driveline 120, the second driveline 140, and pneumatic driveline/tube 172. Balloon pump 110 may be sized and/or shaped to reduce and/or prevent blocking of arteries branching from the aorta, such as the renal arteries. In some embodiments, the balloon pump 110 includes an expandable end effector other than or in addition to a balloon. In some embodiments, the balloon pump 110 may have certain features generally similar to those described in U.S. Pat. Nos. 8,066,628, 8,323,174, and 8,326,421 and U.S. patent application Ser. Nos. 17/944,130, 17/944,127, and 17/944,125, the disclosures of which are incorporated herein by reference in their entireties.

The first driveline 120 may be an elongated structure having a lumen extending therethrough for shuttling gases between the drive unit and the PCAD (i.e., delivering inflation gases to balloon pump 110 and removing inflation gases from balloon pump 110). The first driveline 120 may be able to be positioned at least partially within the patient's vasculature (e.g., between the aorta and an axillary or subclavian artery). After the system 100 is implanted, the first driveline 120 may have a first end portion (e.g., a distal end portion) coupled to the balloon pump 110 and positioned within the patient's vasculature (e.g., within a descending aorta) and a second end portion (e.g., a proximal end portion) coupled to the second driveline 140 and positioned external to the patient's vasculature. The first driveline 120 may exit the patient's vasculature at an arteriotomy in, for example, an axillary artery, a subclavian artery, or another suitable blood vessel.

The second driveline 140 may also be an elongated structure having a lumen extending therethrough. The second driveline 140 may be able to be positioned at least partially subcutaneously but external to the patient's vasculature. After the system 100 is implanted, the second driveline 140 may have a first end portion (e.g., a distal end portion) coupled to the first driveline 120 and a second end portion (e.g., a proximal end portion) coupled to the patient interface device 190. The first driveline 120 and the second driveline 140 may be made of the same or different materials and may have the same or different dimensions (e.g., length, outer diameter, inner diameter, etc.). In some embodiments, the second driveline 140 may include an absorptive feature for reducing longitudinal strain that could otherwise be transferred to the first driveline 120 when the patient moves. For example, in some embodiments the absorptive feature is a curved region (e.g., an “S” shaped or other serpentine curve) that can compress in response longitudinal forces, thereby reducing and/or preventing the longitudinal forces from being transferred to the intravascular portions of the system 100 (e.g., the first driveline 120 and/or the balloon pump 110).

The first driveline 120 may be coupled to the second driveline 140 using any suitable technique. In some embodiments, the first driveline 120 is inserted at least partially into the second driveline 140 (or vice versa) and secured thereto using a compression ring. In some embodiments, the first driveline 120 is sutured, glued, stitched, or otherwise secured to the second driveline 140. In some embodiments, the first driveline 120 is attached to the second driveline 140 via a combination of the foregoing techniques and/or via other suitable attachment techniques. Regardless of the connection mechanism, when the first driveline 120 is coupled to the second driveline 140, the lumen of the first driveline 120 may be fluidly connected to the lumen of the second driveline 140 such that gases that flow through the lumen of the second driveline 140 also flow through the lumen of the first driveline 120. Although described as first and second drivelines, in some embodiments the first driveline 120 and the second driveline 140 may be a single, integral component (i.e., a single driveline). In other embodiments, the second driveline 140 and the pneumatic driveline/tube 172 may be a single, integral component (i.e., a single driveline). In yet other embodiments, the first driveline 120, the second driveline 140, and the pneumatic driveline/tube 172 may be a single, integral component (i.e., a single driveline). Reference herein to a “driveline” may refer to one of the first driveline 120, second driveline 140, and the pneumatic driveline/tube 172, or any combination of any of the above.

As noted above, the first driveline 120 may be coupled to the balloon pump 110 to deliver inflation gases thereto. For example, the first end portion of the first driveline 120 may be connected to the balloon pump 110 such that the lumen extending through the first driveline 120 is in fluid communication with an interior of the balloon pump 110. Accordingly, gas flowing through the lumen of the first driveline 120 towards the balloon pump 110 may flow into the balloon pump 110, causing the balloon pump 110 to transition from the first state to the second state (e.g., causing the balloon pump 110 to inflate). The first driveline 120 may also receive gas from the balloon pump 110 as the balloon pump 110 transitions between the second state and the first state (e.g., as the balloon pump 110 deflates).

The arterial interface device or stopper device 130 may provide long-term (e.g., greater than about 3 months, greater than about 6 months, greater than about 12 months, etc.) hemostasis at an arteriotomy where the first driveline 120 exits the vasculature (e.g., at the subclavian or axillary artery). The stopper device 130 may include a plurality of anchoring elements that may be used to secure the stopper device 130 in a desired orientation or position. In some embodiments, the stopper device 130 may include certain features generally similar to those described in U.S. Pat. No. 7,892,162, the disclosure of which is incorporated herein by reference in its entirety. For example, the stopper device 130 may include a suture ring (e.g., a polyester velour patch sutured to the artery to provide mechanical support), a graft (e.g., a polyester textile defining a lumen and sutured to the suture ring and artery to provider arterial access), and a stopper element (e.g., a silicone plug inserted into the lumen of the graft to provide hemostasis having a lumen that receives the first driveline 120).

The patient interface device 190 may be a transcutaneous device that enables the drive unit 150 to drive operation of the implanted balloon pump 110. For example, in some embodiments the patient interface device 190 provides a stable and/or secure exit site for the second driveline 140, enabling connection of the second driveline 140 directly to the drive unit 150. In other embodiments, the second driveline 140 may be coupled to an internal facing portion of the patient interface device 190, and the drive unit 150 may be coupled to an external facing portion of the patient interface device 190 via tube 172. Tube 172 may be a tube, hose, and/or other conduit having an elongated structure having a lumen extending therethrough. For example, tube 172 may comprise an air tube having a lumen extending therethrough and one or more electrical conduits for serial communication between patient interface device 190 and drive unit 150. In such embodiments, the patient interface device 194) may direct gases received from the drive unit 150 (e.g., via the tube 172) to the second driveline 140 for delivery to the expandable member 110. In some embodiments, the patient interface device 190 may have certain features generally similar to those described in U.S. Pat. No. 10,137,230, the disclosure of which is incorporated herein by reference in its entirety.

The drive unit 150 may generate gas flow into and out of the balloon pump 110 via the first driveline 120, the second driveline 140, and tube 172. For example, the drive unit 150 may generate a positive pressure to accelerate gases into the balloon pump 110 via the first driveline 120, the second driveline 140 and tube 172, thereby inflating the balloon pump 110. The drive unit 150 may also induce a negative pressure to withdraw gases from the balloon pump 110 via the first driveline 120, the second driveline 140, and tube 172, thereby deflating the balloon pump 110. The drive unit 150 may induce gas flow into and out of the balloon pump 110 through a number of different mechanisms. For example, the drive unit 150 may utilize a bellows, a blower, a compressor, an accelerator, or other similar features to direct gas flow into and out of the balloon pump 110. In some embodiments, the drive unit 150 may control the volume of air being pushed into the balloon pump 110 to avoid overinflating the balloon pump 110. For example, in an embodiment utilizing a bellows to generate air flow, the volume of airflow generated by the bellows (e.g., the volume of the bellows) may be matched to an interior volume of the balloon pump 110. In some embodiments, the drive unit 150 uses ambient air from the environment surrounding the drive unit 150 (e.g., “room air”) to drive operation of the system 100. Without being bound by theory, using ambient air is expected to reduce the size, weight, and/or cost of the drive unit 150 relative to a drive unit that relies on an internal gas or fluid supply (e.g., helium tanks). For example, in some embodiments the drive unit 150 may weigh about 2.2 kg or less. Accordingly, in some embodiments the drive unit 150 may be portable/ambulatory. In other embodiments, the drive unit 150 may be operably coupled to or otherwise include a gas supply, such as helium tanks (not shown). The driveline 140 or tube 172 may be disconnected near the patient interface device 190 when the system 100 is not being actively used. In some embodiments the drive unit ISO may have certain features generally similar to those described in U.S. patent application Ser. No. 17/878,632, the disclosure of which is incorporated herein by reference in its entirety.

The sensors 160 may sense one or more physiological parameters related to the patient's native heart rhythm to synchronize operation of the system 100 with the cardiac cycle. In particular, one or more sensed physiological parameters may be used to automatically synchronize operation of the drive unit 150 with the patient's native heartbeat to ensure the balloon pump 110 is being inflated and deflated at appropriate times during the cardiac cycle. In some embodiments, the sensors 160 may sense the one or more physiological parameters in real time. As illustrated in FIG. 1, the sensors 160 may be coupled to the patient interface device 190 via wires 162. Wires 162 may be intracardiac, subcutaneous, supercutaneous, or any combination of the foregoing. The patient interface device 190 may relay the sensor data (e.g., data received from the sensors 160) to the drive unit 150 via a wired or wireless connection. In other embodiments, the sensors 160 may be connected, via a wired or wireless connection, to the drive unit 150 and can transmit sensor data directly to the drive unit 150 without using the patient interface device 190. The sensors 160 may be implanted sensors, external sensors, or any combination of the foregoing. For example, in some embodiments, the sensors 160 may be implanted bipolar electrodes positioned at and/or proximate the heart or other appropriate tissue to determine, for example, when the left ventricle is contracting or relaxing. Sensors 160 may deliver to the drive unit 150, one or more of the following: the heart's electrical signal(s)(e.g., signals which may be processed and displayed on a monitor or printer as an ECG signal), pressure information associated with the pressure proximate the balloon pump 110 (e.g., proximate the proximal end of the balloon pump 100), ambient noise information (e.g., noise associated with the pectoral muscle, electronic noise from the system 100 or from other devices (not depicted) associated with or in proximity to the patient, etc.), noises made by the heart during a cardiac cycle (e.g., information that may be processed and displayed on a monitor or printer as an phonocardiograph signal), other pressure signals, etc. In some embodiments, sensors 160 are electronic sound transducers, pressure transducers, piezoelectric transducers or other transducers or sensors suitable for making physiological parameter measurements. Although FIG. 1 depicts three sensors 160, system 100 may incorporate less or more than three sensors 160 depending on the nature of what is being sensed in any particular embodiment.

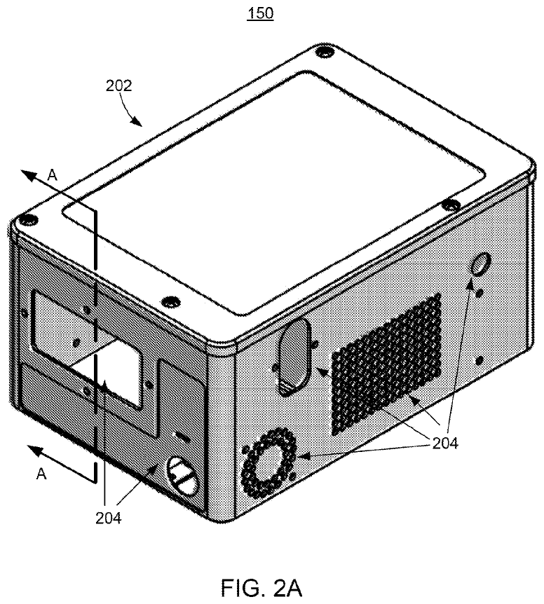

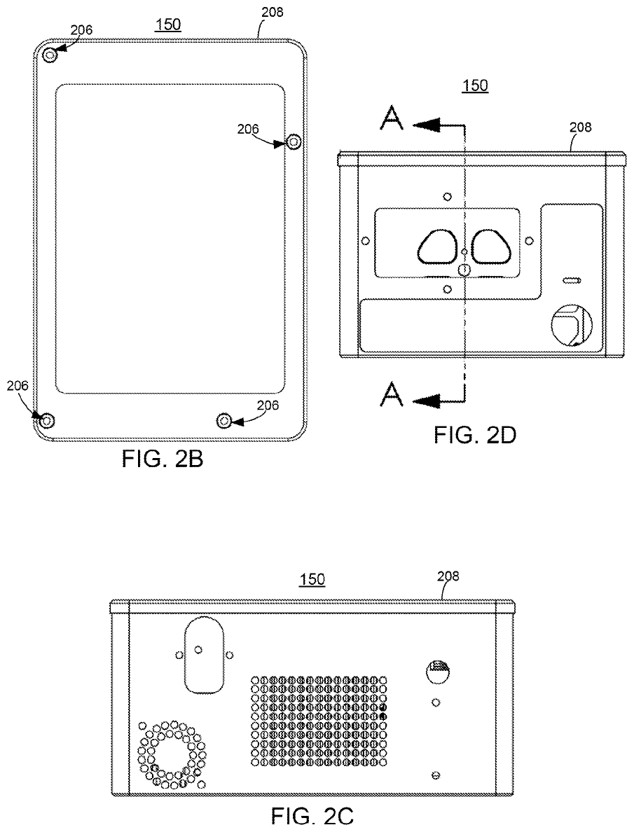

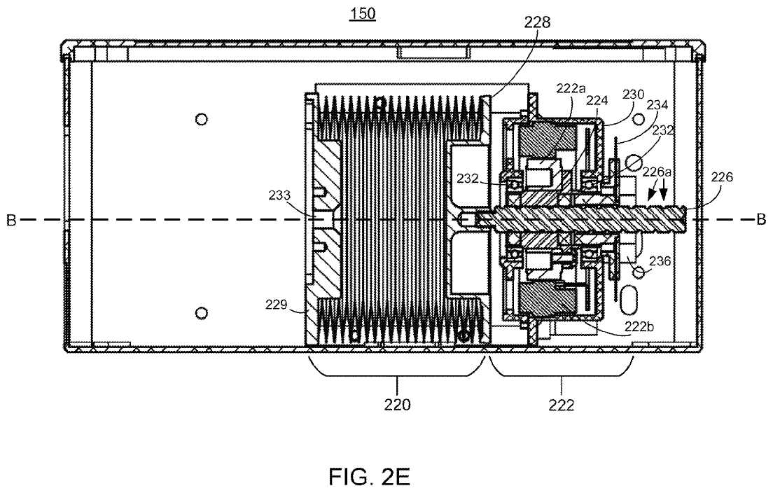

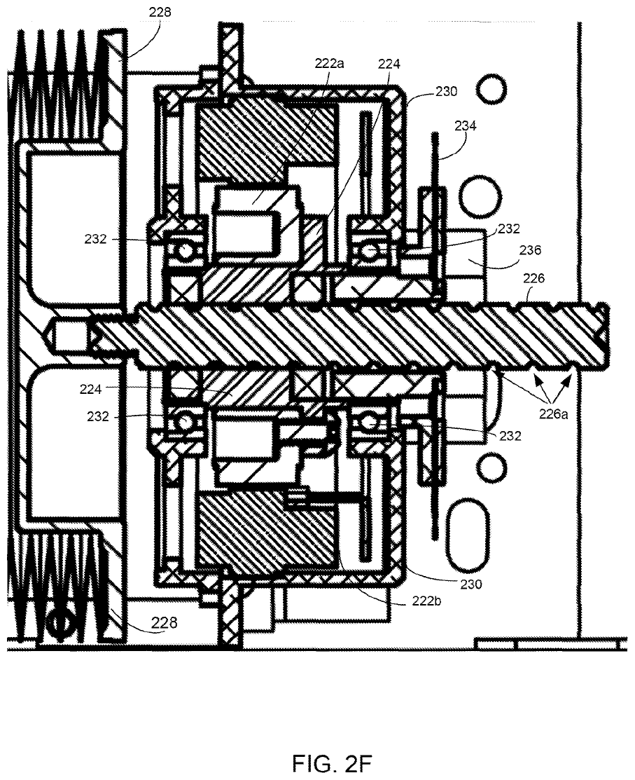

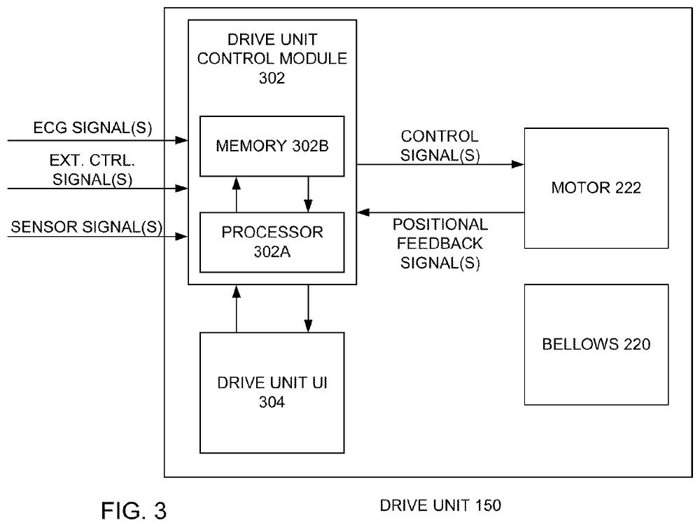

An exemplary drive unit 150 is depicted in FIGS. 2A-2F. Drive unit 150 may include a case 202 having various ports 204, an affixable top plate 208 and fasteners 206 for closing the case 202. Ports 204 may be used for, among other things, display readouts, power buttons, ventilation, and connectivity to other system components (e.g., pneumatic driveline/tube 172). Case 202 may house a bellows 220 and motor 222 (e.g., an electric motor such as a brushless DC motor. Case 202 may further house other components such as electrical components used to control motor 222 and a manifold (such as manifold 502 described below) used to couple the bellows output/pneumatic output 223 to communicate gas from the bellows 220 to a driveline of a PCAD system (e.g., one or more of drivelines 172, 140, 120 and PCAD system 100). In some embodiments, such components may be housed in the space to the left of bellows 220 in FIG. 2E. The electrical components may include a drive unit control module 302 (described below).

Bellows 220 may be an axial expansion bellows that is able to expand and contract along the B-B axis depicted in FIG. 2E in response to rotation of the motor 222 in different directions. In the embodiment depicted by FIGS. 2E and 2F, the bellows 220 is expanded such that the PCAD (e.g., balloon pump 110) is deflated. When the bellows 220 is compressed by motor 222, the PCAD (e.g., balloon pump 110) is inflated. The bellows 220 may have a constant cross-sectional geometry along its length so that the volume within the bellows 220 is varied according to the bellows length.

The motor 222 may include a rotor 222a and stator 222b. The rotor 222a may be joined to a rotary-to-linear transformer. For example, the rotor 222a may be joined to a ball nut 224 carrying a ball screw 226 that is affixed to a dynamic flange 228. The bellows 220 may be sealed by the dynamic flange 228 at a first bellows end proximal to the ball screw 226 and by a static flange 229 at a second bellows end that is distal to the ball screw 226. A bellows outlet 233 may allow communication of gas within the driveline (e.g., pneumatic driveline/tube 172, second driveline 140, and/or first driveline 120) to the PCAD (e.g., balloon pump 110) in response to movement of the bellows 220.

In some embodiments, the bellows outlet 233 may be formed within the static flange 229. The ball screw 226 in combination with the ball nut 224 may form a mechanical rotary-to-linear transformer that converts rotational motion of the motor 222 to linear motion with little friction. Rotation of the ball nut 224 by the rotor 222a within the stationary stator 222b may cause the ball screw 226 to move linearly along axis B-B and, correspondingly, cause linear movement of the bellows 220.

The ball screw 226 may be threaded to provide a helical raceway 226a for balls (not depicted) of the ball nut 224 and may function as a precision screw.

The rotor 222a and ball nut 224 assembly may be mounted to a housing 230 of the motor 222 via radial bearings 232. The inner race of the radial bearings 232 may be affixed to the rotor 222a and ball nut 224 assembly while the outer race of the radial bearings 232 and the stator 222b may be affixed to a motor housing 230. Actuation of the motor 222 may cause rotational movement of the rotor 222a and ball nut 224 which may cause balls (not depicted) of the ball nut 224 to ride within the helical raceway 226a of the ball screw 226 and convert the rotational movement of the rotor 222a into linear movement of the ball screw 226 along axis B-B (FIG. 2E). Movement of the ball screw 226 is translated to the bellows 220 via the dynamic flange 228, causing the bellows 220 to expand or contract to create negative or positive fluid flow, respectively, within the driveline (e.g., pneumatic driveline % tube 172, second driveline 140, and/or first driveline 120), which is in fluid connection with the PCAD (e.g., balloon pump 110) via the bellows outlet 233. In some embodiments, the ball screw 226 may be fixedly mated to the dynamic flange 228 via a threaded interface (as depicted in FIGS. 2E and 2F). In some embodiments, the dynamic flange 228 and ball screw 228 are one continuous piece. When the ball screw 226 is fixedly mated to the dynamic flange 228 or when the ball screw 226 and dynamic flange 228 are one continuous piece, ball screw 228 may directly and efficiently translate its motion to the bellows (e.g., withdrawal of the ball screw 226 away from the static flange 229 may pull the dynamic flange 228 to expand the bellows 220).

With reference to FIGS. 2E-F, and 3, drive unit 200 may include drive unit control module 302 operative to control motor 222. Drive unit control module 302 may include any suitable logic modules. In one embodiment, drive unit control module 402 includes a processor 302A and a memory 302B. Processor 302A may include one or more dedicated or non-dedicated micro-processors, micro-controllers, sequencers, micro-sequencers, digital signal processors, processing engines, hardware accelerators, applications specific circuits (ASICs), state machines, programmable logic arrays, any integrated circuit(s), discrete circuit(s), etc. that is/are capable of processing data or information, or any suitable combination(s) thereof. Memory 302B may include any suitable non-volatile memory device, chip, or storage device capable such as one or more of: system memory, frame buffer memory, flash memory, random access memory (RAM), read only memory (ROM), a register, and a latch. Processor 302A may be capable of executing executable instructions (e.g., as stored in memory 302B). Processor 302A may be configured to control motor 222, and by extension, the bellows 220.

For example, an encoder disk 234 and encoder sensor 236 may determine the angular position, speed, and/or direction of the rotor and provide such information as positional feedback signal(s) to drive unit control module 302. In other embodiments, a linear position sensor (not depicted) may be used to determine the position of the ball screw 226 or bellows 220 and to provide such positional feedback signal(s). Drive unit control module 302 may also receive control information from drive unit user interface (UI) 304 and provide display information (e.g., status information) to drive unit UI 304. Relatedly, drive unit control module 302 may similarly receive ECG signal(s) from skin interface device 190 and one or more sensors 160, one or more external control signals (e.g., from a tablet or other computing device (not depicted)), and one or more sensor signals. For example, drive unit control module 203 may receive a pressure signal as observed by a pressure sensor (not depicted) located in proximity to PCAD (e.g., balloon 110) when disposed in an artery (e.g., the descending aorta, as depicted in FIG. 1) of a patient undergoing therapy (e.g., counterpulsation therapy). The one or more pressure signals may be, indicative of the pressure exerted on PCAD (e.g., balloon pump 110) in such artery and/or the pressure within such artery.

Drive unit control module 302 may use one or more of the positional feedback signals, drive unit UI control signals, ECG signals, external control signals, and sensor signals to control motor 222. For example, ECG signals may be used to ensure proper timing of the inflation and/or deflation of the PCAD (e.g., balloon pump 110), for example to pursue counterpulsation. And drive unit UI control signal and external control signals may be used to change the volume displacement and/or support ratio (e.g., 1:1, 1:2, 1:3, etc. support). Without being bound by theory, gradually reducing the volume displacement of the PCAD (e.g., balloon pump 110) over time may result in a controlled loading of the heart which in some instances may be beneficial for cardiac recovery. A support ratio measures the ratio of beats to inflations of the PCAD (e.g., balloon pump 110). For example, a 1:1 support ratio indicates the for every beat there is a corresponding inflation of the PCAD, whereas a 1:2 support ratio indicates that there are two beats before each inflation, and a 1:3 support ratio indicates that there are three beats before each inflation.

Other embodiments may employ linear brushless DC motors, solenoids, and/or piezoelectric actuators to compress and expand bellows 220.

Drive unit 150 may be used to effectively inflate and deflate PCAD (e.g., balloon pump 110) using air to provide counterpulsation, copulsation or other therapy in patients with heart failure or other heart disease.

### Leak Detection Theory

Because no fluid should be entering or leaving the PCAD system 100, the pressure in the system 100 can be monitored to determine if there is a leak. However, simply comparing pressures at identical points in the cycle (e.g., pumping cycle) may not accurately determine if there is a leak, because a change in temperature may result in a commensurate change in pressure according to the Pressure-Temperature Gas Law.

The Pressure-Temperature Gas Law is also known as Gay-Lussac's Law and it states that the pressure of a given mass of a gas varies directly with the absolute temperature of the gas when the volume is kept constant. The mathematical expression for the Pressure-Temperature Gas Law is set forth below as Equation 1 where P₁,abs is the absolute pressure at a first time, T₁,abs is the absolute temperature at the first time, P₂,abs is the absolute pressure at a second time, and Tz,abs is the absolute temperature at the second time. Absolute pressures are pressures expressed on an absolute pressure scale. An absolute pressure scale relates zero pressure to the pressure in the empty, air-free space of the universe. In other words, an absolute pressure is the pressure measured with reference to an ideal or absolute vacuum or no atmospheric pressure. The units of absolute pressure can be any suitable unit such as millimeters of mercury (mmHg), atmospheres (atm). Pascals, (Pa), pounds per square inch absolute (psig), etc. Absolute temperatures are temperatures expressed on an absolute temperature scale.

An absolute temperature scale relates zero temperature to the coldest temperature at which there is no internal energy and relevant particles are stationary. The Kelvin scale and the Rankine scale are examples of absolute temperature scales.

$$\frac{{P}_{1 , abs}}{{T}_{1 , abs}} = \frac{{P}_{2 , abs}}{{T}_{2 , abs}} \qquad ( Equation 1 )$$

Equation 1 can be readily manipulated to equate the ratio of the first absolute pressure/first absolute temperature ratio to the ratio of an expected change in pressure/actual change in temperature over a period of time (e.g., from a first time to a second time), which is set forth in Equation 2, where ΔP_{expected} may be expressed on the same absolute pressure scale as P₁,abs or a complementary pressure scale and ΔT_{actual} may be expressed on the same absolute temperature scale as T₁,abs or a complementary temperature scale. A complementary pressure scale is one where a unit change on such complementary pressure scale is equal to a unit change on the absolute pressure scale. For example, if P₁,abs is measured in absolute mmHg, then ΔP_{expected} may be expressed either in absolute mmHg or mmHg relative to atmospheric pressure. A complementary temperature scale is one where a unit change on such complementary temperature scale is equal to a unit change on the absolute temperatures scale. For example, if T₁,abs is measured in Kelvin, then ΔT_{actual} may be expressed either in Kelvin or Celsius because the Kelvin scale is merely a shifted version of the Celsius scale (i.e., a one degree change in Celsius results in a 1 Kelvin change). In this example where T₁,abs is measured in Kelvin, ΔT_{actual} may not be expressed in Fahrenheit because a unit degree change in Fahrenheit does not correspond to a 1 Kelvin change.

As used herein, “expressed in” when used in connection with a particular scale means, as the case may require, measured on such scale or calculated or determined in units of such scale.

$$\frac{Δ {P}_{expected}}{Δ {T}_{actual}} = \frac{{P}_{1 , abs}}{{T}_{1 , abs}} \qquad ( Equation 2 )$$

Because ΔT_{actual} represents the change in temperature over the period of time (e.g., from a first time to a second time), it can be expressed as (T₂−T₁) and substituted into Equation 2 to provide an expression that predicts the expected change in pressure for an actual or known change in temperature over that period of time of operation of a PCAD system. The period of time may be the time starting at the first time and ending at the second time. Such an expression is set forth as Equation 3, where subscripts represent expected (if noted) or actual (where not noted) measures of pressure at the first time (subscript “1”) or the second time (subscript “2”) and where each of T₂ and T₁ are actual measurements expressed on the same absolute temperature scale as T₁,abs or a complementary temperature scale.

$$\frac{Δ {P}_{expected}}{{T}_{2} - {T}_{1}} = \frac{{P}_{1 , abs}}{{T}_{1 , abs}} \qquad ( Equation 3 )$$

One can readily solve Equation 3 for ΔP_{expected} as is indicated in Equation 4.

$$Δ {P}_{expected} = \frac{{P}_{1 , abs}}{{T}_{1 , abs}} ( {T}_{2} - {T}_{1} ) \qquad ( Equation 4 )$$

In the case of Equation 4, ΔP_{expected} is a temperature dependent pressure change that represents an expected pressure difference in the PCAD system over the period of time, due or attributable to the change in temperature, i.e., the difference between T₂ and T₁. Accordingly, subtracting the temperature dependent pressure change, ΔP_{expected}, from the actual pressure measured at the second time, P₂ where P₂ is an actual measurement expressed on the same absolute pressure scale as P_{1,abs} or a complementary pressure scale, gives a temperature-compensated pressure at the second time that accounts for the temperature dependent pressure change given the change between T₂ and T₁. The temperature-compensated pressure reading, [P₂−ΔP_{expected}] should be equivalent to P₁ if there is no leak in the PCAD system. This is indicated in Equation 5, where for clarity each of P₂ and P₁ are on the same pressure scale, either the same absolute pressure scale as P_{1,abs} or a complementary pressure scale. If there is a leak in the PCAD system, the temperature-compensation pressure reading, [P₂−ΔP_{expected}] will be less than or more than insubstantially less than P₁. This is indicated in Equation 6. In one embodiment. “insubstantially less” may be 1%, 2%, 3%, 4%, 5%, 6%, 7%, 8%, 9% or 10% of the pressure of the PCAD at the first time, P₁. In another embodiment, “insubstantially less” may be any amount determined by a treating medical professional to ensure effective CAD therapy. In another embodiment, “insubstantially less” may correspond to a fatal or harmful amount of leakage into a patient.

$$[ {P}_{2} - Δ {P}_{expected} ] - {P}_{1} = 0 ( No leak ) \qquad ( Equation 5 )$$

$$[ {P}_{2} - Δ {P}_{expected} ] < {P}_{1} ( Leak determined ) \qquad ( Equation 6 )$$

Returning to Equation 3, ΔP_{expected} can be expressed as P_{2,expected}−P₁ and substituted therefor to provide an expression that predicts the expected pressure at the second time for an actual or known change in temperature over the period of time of operation of a PCAD system, where P_{2,expected} and P₁ are on the same pressure scale, either the same absolute pressure scale as P_{1,abs} or a complementary pressure scale and where P₁ is an actual measurement. After substitution, this equation can readily be solved for P_{2,expected} as is indicated in Equation 7.

$${P}_{2 , expected} = [ \frac{{P}_{1 , abs}}{{T}_{1 , abs}} ( {T}_{2} - {T}_{1} ) ] + {P}_{1} \qquad ( Equation 7 )$$

In the case of Equation 7, P_{2,expected} is the expected pressure at the second time due or attributable to the change between T₂ and T₁. The actual pressure measured at the second time, P₂ should be equivalent to the expected pressure at the second time, P_{2,expected} if there is no leak in the PCAD system, where P₂ is on the same pressure scale as P_{2,expected} and P₁. This is indicated in Equation 8. If there is a leak in the PCAD system, the measured pressure at the second time, P₂ will be less than or more than insubstantially less than expected pressure at the second time, P_{2,expected}. This is indicated in Equation 9. In one embodiment, “insubstantially less” may be 1%, 2%, 3%, 4%, 5%, 6%, 7%, 8%, 9% or 10% of the expected pressure of the PCAD at the second time, P_{z,expected}. In another embodiment, “insubstantially less” may be any amount determined by a treating medical professional to ensure effective CAD therapy. In another embodiment, “insubstantially less” may correspond to a fatal or harmful amount of leakage into a patient.

$${P}_{2} = {P}_{2 , expected} ( No leak ) \qquad ( Equation 8 )$$

$${P}_{2} < {P}_{2 , expected} ( Leak determined ) \qquad ( Equation 9 )$$

### Leak Detection Apparatus

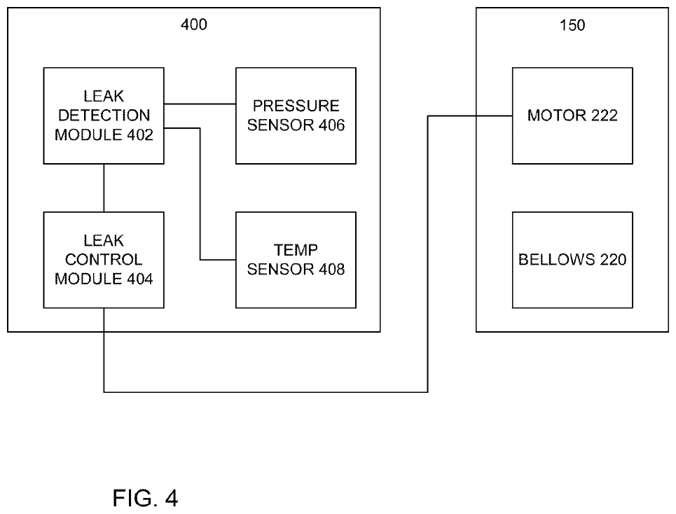

FIG. 4 illustrates an exemplary leak detection apparatus 400 comprising leak detection module 402 and leak control module 404. Leak detection apparatus 400 may optionally include pressure sensor 406 and temperature sensor 408. Leak detection apparatus 400 may be configured to implement the foregoing leak detection theory associated with the Pressure-Temperature Gas Law in connection with a PCAD system, such as PCAD system 100 and its component parts, including for example, drive unit 150, driveline (e.g., first driveline 120, second driveline 140, and/or pneumatic drive line/tube 172) and PCAD 110.

Leak detection module 402 may be configured to determine if or that there is a leak in a PCAD system over a period of time defined by a first time and a second time. The determination may be based on a first PCAD system pressure at the first time (P1), a first PCAD system temperature at the first time (T1), a second PCAD system pressure at the second time (T1) and a second PCAD system temperature at the second time (T2). The determination if or that there is a leak in the PCAD system may include accounting for a change in the PCAD system pressure over the period of time attributable to the difference between T2 and T1.

In one embodiment, the first and second pressures may be obtained from pressure sensor 406. In another embodiment, the first and second temperatures may be obtained from temperature sensors 408. The first and second pressures and the first and second temperatures may be associated with at least one component of the PCAD system. In another embodiment, the first and second pressures and the first and second temperatures may be associated with the same component of the PCAD system. In yet another embodiment, the first and second pressures are associated with a single first component of the PCAD system and the first and second temperatures are associated with a single second component of the PCAD system. In other embodiments, the first pressure and the first temperature are associated with a single third component of the PCAD system and the second pressure and the second temperature are associated with a single fourth component of the PCAD system. In one embodiment, the first and second pressures and the first and second temperatures are associated with a manifold of the drive unit 150.

In some embodiments, the first and second times are determined to be when the pneumatic volume of the PCAD system at the second time is expected (e.g., absent a leak) to be the same or substantially the same as the pneumatic volume of the PCAD system at the first time, and/or when the gas in the PCAD system is in a steady state. Substantially the same may be 1%, 2%, 3%, 4%, 5%, 6%, 7%, 8%, 9% or 10% of the pneumatic volume associated with the first time. For PCADs having drive units such as drive unit 150, the pneumatic volume at the second time may be determined to be the same or substantially the same as the pneumatic volume at the first time when the drive unit has caused the PCAD, e.g., balloon pump 110 to be in its most deflated state. In embodiments where the drive unit 150 has a bellows mechanism to inflate or partially inflate and deflate or partially deflate the PCAD, this most deflated state may correspond to when the bellows mechanism is in its most expanded position.

In some embodiments, it may be desirable to pause operation of the PCAD system at or approximately at points of operation of the PCAD system corresponding to when the pneumatic volumes of the PCAD system at the second and first times are expected to be the same or substantially the same. Stopping operation may allow the air to equilibrate before taking the actual pressure measurements P1 and P2. Operation may continue after the measurements have been taken.

In some embodiments, it may be desirable to record the actual pressure measurements P1 and P2 when the pressure measurements are steady or substantially steady. In some embodiments, this may include monitoring the readings of P1 and P2 at points of operation of the PCAD system corresponding to when the pneumatic volumes of the PCAD system at the second and first times are expected to be the same or substantially the same. When the pressure readings (e.g., P1 or P2, as the case may be) have become steady or substantially steady, they may be taken as P1 and P2. Pressure reading may be considered steady when they have not moved over a determined period of time, and they may be considered substantially steady when they do not change more than 1%, 2%, 3%, 4%, 5%, 6%, 7%, 8%, 9%, or 10% within a period of time. In some embodiments the period of paused operation can be a function of the length of time to allow the pressure readings to become steady or substantially steady.

In some embodiments, the leak detection module 402 accounts for the change in PCAD system pressure attributable to the difference between T1 and T2 by determining a temperature dependent pressure change at the second time based on the difference between T1 and T2. The temperature dependent pressure change may represent an expected difference in the PCAD system over the period of time given the change in T1 and T2. In one embodiment, the temperature dependent pressure change may be defined in accordance with Equation 4 as the product of (a) P1 on an absolute pressure scale (P1, ABS) over T1 on an absolute scale (T1, ABS) and (b) the difference in T2 and T1, where T1 and T2 are expressed on the same temperature scale, such scale being either the same absolute temperature scale as T1, ABS or a complementary temperature scale.

Leak detection module 402 may further be configured to determine a temperature-compensated pressure at the second time based on P2 and the temperature dependent pressure change. This temperature-compensated pressure at the second time may be defined as P2 reduced by the temperature dependent pressure change, where P2 is an actual measurement expressed on the same absolute pressure scale as P1, ABS or a complementary scale.

The leak detection module 402 may also be configured to determine if there is a leak in the PCAD system in accordance with Equations 5 and 6. In particular, leak detection module 402 may determine if there is a leak or that there is a leak when the temperature-compensated pressure is less than or more than insubstantially less than P1, where P1 is an actual measurement on the same pressure scale as P₂.

In other embodiments, the leak detection module 402 accounts for the change in PCAD system pressure attributable to the difference between T1 and T2 by determining an expected PCAD system pressure at the second time based on the difference between T1 and T2. The expected PCAD system pressure at the second time may be defined in accordance with Equation 7 above as the sum of P1 and the product of (a) P1 on an absolute pressure scale (P1, ABS) over T1 on an absolute scale (T1, ABS) and (b) the difference in T2 and T1, where P1 is expressed on either the same absolute pressure scale as P1, ABS or a complementary pressure scale and T1 and T2 are expressed on the same temperature scale, such scale being either the same absolute temperature scale as T1, ABS or a complementary temperature scale.

Next, the leak detection module 402 may compare the expected PCAD system pressure at the second time with P2, where P2 is expressed on the same scale as the expected PCAD system pressure and P1. Leak detection module 402 can determine if there is a leak in the PCAD system (or that there is a leak in the PCAD system) when P2 is less than or more than insubstantially less than the expected PCAD system pressure. This comparison and determination is consistent with Equations 8-9.

In some embodiments, it may be necessary to convert one or more of the first pressure, second pressure, first temperature, and second temperature from either an absolute scale or a complementary temperature scale. For example, if pressure sensor 406 is a gauge sensor (i.e., it produces pressure readings relative to atmospheric temperature), it may be necessary to convert relative units to absolute units as is contemplated in the above discussion. Similarly, if temperature sensor 408 produces temperature readings in degrees Fahrenheit, it may be necessary to convert such units to Kelvin or Celsius, as is contemplated in the above discussion. Leak detection module 402 may include logic to make such conversions.

The leak control module 404 may be configured to control the PCAD system, based on or in response to a determination that there is a leak in the PCAD system by the leak detection module 402. For example, leak control module 404 may be configured to cause the PCAD system and in particular the applicable drive unit to cease actuation of the PCAD system (e.g., by ceasing actuation of the drive unit itself and thereby ceasing actuation of the PCAD) in response to the determination that there is a leak in the PCAD system.

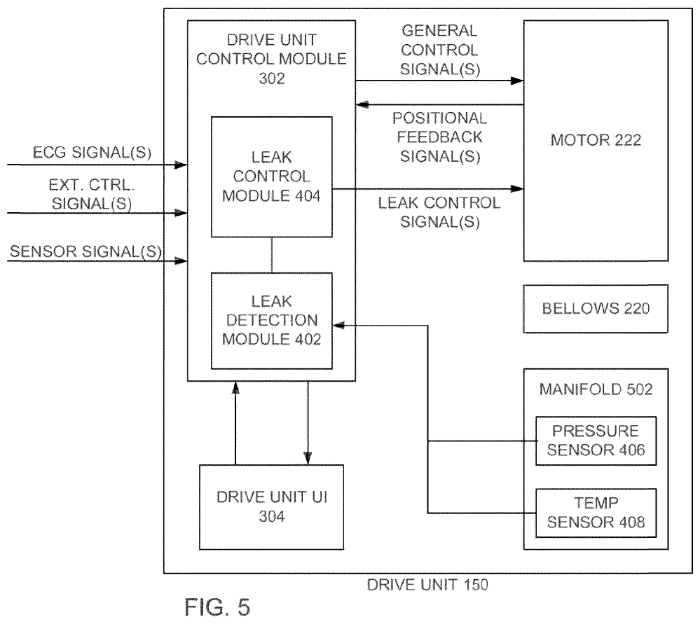

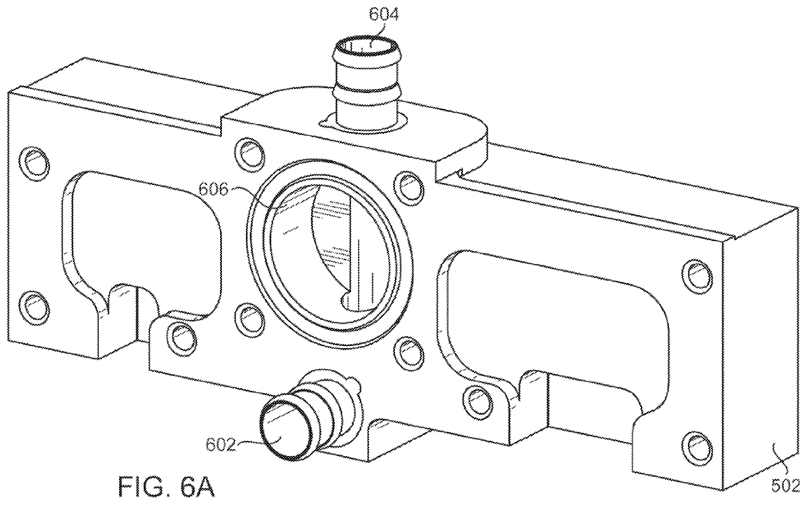

FIG. 5 illustrates components of the leak control apparatus 400 as integrated within the exemplary drive unit 150. Leak detection control module 402 and leak detection module 404 may be components of or otherwise associated with the drive unit control module 302 and optional pressure sensor 406 and optional temperature sensor may be associated with manifold 502 (described below).

### Exemplary Manifold

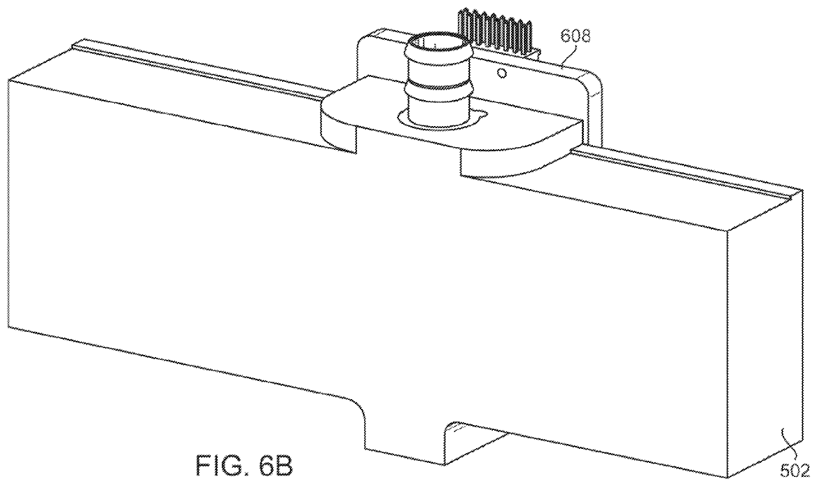

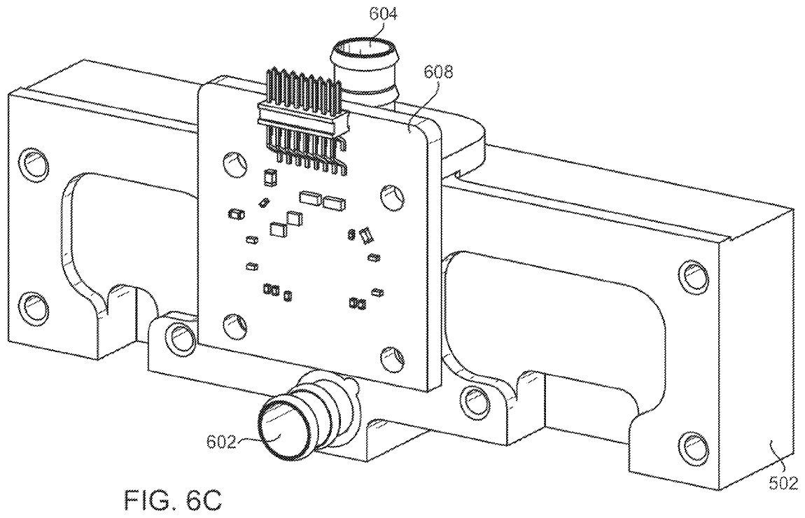

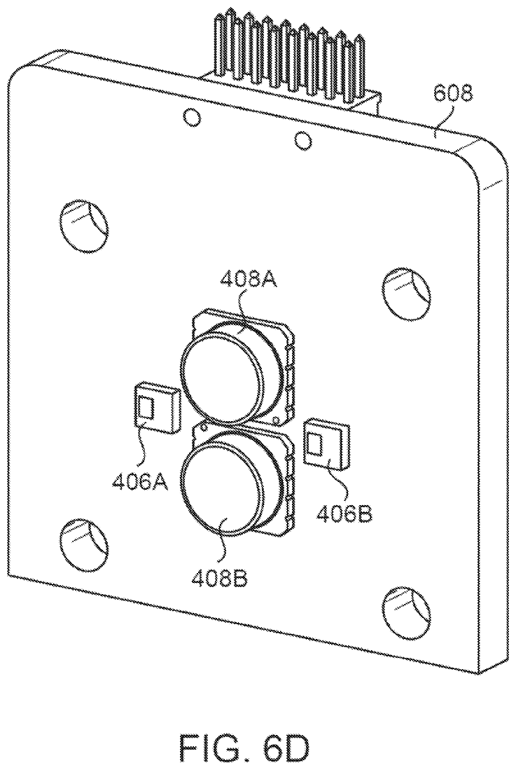

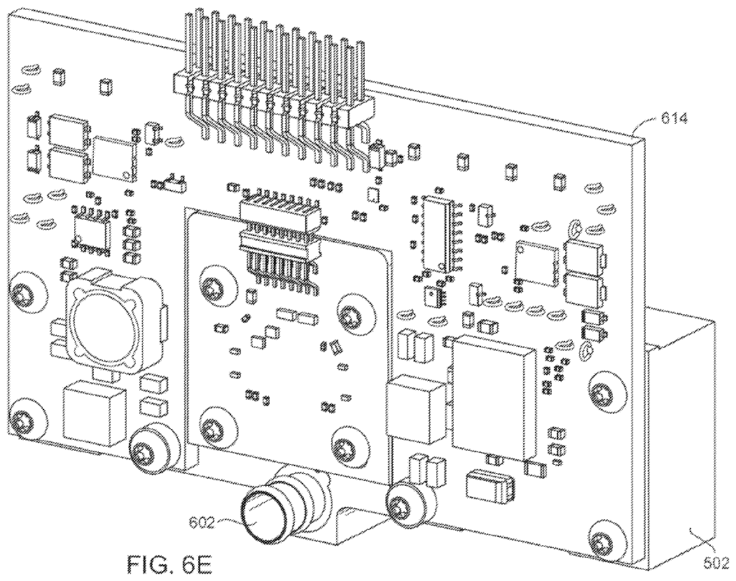

FIGS. 6A-6E illustrate an exemplary manifold 502. As described above, the manifold 502 may occupy the space to the left of bellows 220 in the exemplary drive unit 150 depicted in FIG. 2E along with other electrical components used to control motor 222. Manifold 502 may include an inlet 602 which may be configured as a hose barb connector that is coupled to the output of the bellow 220. For example, the inlet 602 may be a hose barb connector that is coupled directly or indirectly (e.g., via a short tube) to bellows outlet 233. Manifold 502 may further include an outlet 604 which may be configured as a hose barb connector that is coupled to the driveline. For example, outlet 604 may be a hose barb connector that is coupled directly or indirectly (e.g., via a short tube) to pneumatic driveline/tube 172 or any other component of the PCAD system driveline. Manifold 502 may include a manifold cavity 606 that is in fluid communication with the inlet port 602 and 604.

Inlet 602 and outlet 604, including manifold cavity 606 may form a manifold valve to communicate air from bellows 220 to driveline. In some embodiments, the manifold valve may ensure that the air from bellows 220 is evenly distributed as it is communicated to the driveline. One of skill in the art will appreciate that driveline 150 may be configured to use different gases other than air and that such drivelines may use a different mechanism to compress such gas. Manifold 502 may be employed for similar purposes in such configurations.

some embodiments, pressure sensor 406 and temperature sensor 408 may be disposed within the manifold cavity (06. For example, one or more pressure sensors 406A and 406B and one or more temperatures sensors 408A and 4088 may be disposed on printed circuit board 608 that is disposed over and configured to seal the manifold cavity 606. Circuitry on the printed circuit board 608 may communicate pressure and temperature readings to the leak detection module 402.

In some embodiments, printed circuit board 608 is a daughter board that is in electrical communication with a mother printed circuit board (or mother board) 614. The mother board 614 may include one or more of drive unit control module 302, leak detection module 402 and leak control module 404.

### Method for Leak Detection

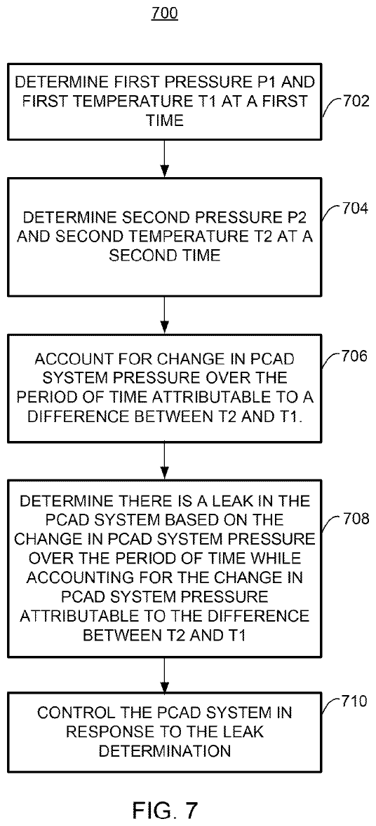

FIG. 7 illustrates an exemplary method 700 for using a leak detection apparatus such as the leak detection apparatus of FIGS. 4 and 5 to determine there is a leak in a PCAD system, such as the PCAD system depicted in FIG. 1. The method 700 may optionally include block 702 where a first pressure P1 and a first temperature T1 is determined at a first time. In one embodiments, P1 and T1 are determined or measured using pressure sensor 406 and temperature sensor 408. The method 700 may also include block 704 where a second pressure P2 and a second temperature T2 is determined at a second time. In some embodiments. P2 and T2 may be determined or measured using pressure sensor 406 and temperature sensor 408. Method 700 may also include block 706 where a change in PCAD system pressure is accounted for over the period of time attributable to a difference between T2 and T1, where P1, T1, P2, and T2 are associated with at least one component of a PCAD system. Block 706 may be performed using leak detection 402 and the Pressure-Temperature Gas Law described above. At block 708, method 700 may determine there is a leak in the PCAD system based on the change in PCAD system pressure over the period of time, while accounting for the change in PCAD system pressure over the period of time attributable to the difference between T2 and T1. Like block 706, block 708 may be implemented using leak detection module 402 and the Pressure-Temperature Gas Law. Finally, method block 710 controls the PCAD system 100 in response to the detected leak. For example, the method 700 may cause leak control module 404 to issue an alarm or stop operation of the PCAD system 100.

The technology and techniques described herein offer efficient and effective detection of leaks during operation of PCADs, including but not limited to counterpulsation devices. Because changes in some environmental conditions, such as temperature, may influence pneumatic pressure, pressure measurements alone may not be a reliable indicator of a PCAD system leak. Rather, pressure changes caused by changes in temperature must be accounted for to detect a leak and control a PCAD system in response to or based on such detection.

Although the foregoing technology was described with reference to counterpulsation devices in particular, the technology may be readily applied to other PCADs, including those operating in copulsation or other therapeutic modalities.

### EXAMPLES OF SOME OF THE EMBODIMENTS OF THE DISCLOSURE

Example 1. An apparatus comprising: a leak detection module operative to determine if there is a leak in a pneumatic cardiac assist device (PCAD) system over a period of time defined by a first time and a second time, wherein: the determination is based on a first PCAD system pressure at the first time (P1), a first PCAD system temperature at the first time (T1), a second PCAD system pressure at the second time (P2), and a second PCAD system temperature at the second time (T2), while accounting for a change in PCAD system pressure over the period of time attributable to a difference between T2 and T1, and P1, T1, P2, and T2 are associated with at least one component of the PCAD system; and a leak control module that is operative to control the PCAD system in response to a determination that there is a leak in the PCAD system by the leak detection module.

Example 2. The apparatus of example 1, further comprising: a pressure sensor operative to determine P1 and P2; and a temperature sensor operative to determine T1 and T2.

Example 3. The apparatus of example 2, wherein: the PCAD system includes a pneumatic drive unit, a driveline, and the PCAD, the pneumatic drive unit is operative to generate gas pressure and gas flow to operate the PCAD, the driveline is operative to shuttle the gas from the pneumatic drive unit to the PCAD, and the PCAD is operative to provide therapeutic care to a patient.

Example 4. The apparatus of any of examples 2-3, wherein the PCAD is one of: an intra-aortic balloon, a patch device, a cuff device, and a pump device.

Example 5. The apparatus of any of examples 3-4, wherein the pneumatic drive unit comprises a manifold that is operative to communicate gas to the driveline, wherein: P1 and P2 are the pressure of gas within the manifold at the first and second times, respectively, and T1 and T2 are the temperature of gas within the manifold at the first and second times, respectively.

Example 6. The apparatus of any of examples 3-5, wherein the gas is air.

Example 7. The apparatus of any of examples 1-6, wherein the leak detection module determines a leak using the Pressure-Temperature Gas Law.

Example 8. The apparatus of any of examples 1-7, wherein the leak detection module is operative to account for the change in PCAD system pressure attributable to the difference between T1 and T2 by determining a temperature dependent pressure change at the second time based on the difference between T1 and T2, wherein the temperature dependent pressure change represents an expected difference in the PCAD system over the period of time given the change in T1 and T2.

Example 9. The apparatus of example 8, wherein: the temperature dependent pressure change is defined as the product of (a) P1 on an absolute pressure scale (P1, ABS) over T1 on an absolute temperature scale (T1, ABS) and (b) the difference in T2 and T1, and T1 and T2 are expressed on the same temperature scale, said same temperature scale being one of the absolute temperature scale and a complementary temperature scale.

Example 10. The apparatus of any of examples 8-9, wherein the leak detection module is operative to convert P1 into P1, ABS and to convert T1 into T1, ABS.

Example 11. The apparatus of any of examples 8-10, wherein the leak detection module is further operative to determine a temperature-compensated pressure at the second time based on P2 and the temperature dependent pressure change.

Example 12. The apparatus of example 11, wherein the temperature-compensated pressure is defined as P2 reduced by the temperature dependent pressure change.

Example 13. The apparatus of any of examples 11-12, wherein the leak detection module is further operative to determine that there is a leak in the PCAD system when the temperature-compensated pressure is one of: less than P1 and more than insubstantially less than P1.

Example 14. The apparatus of any of examples 1-13, wherein the leak detection module is operative to account for the change in PCAD system pressure attributable to the difference between T1 and T2 by determining an expected PCAD system pressure at the second time based on the difference between T1 and T2.

Example 15. The apparatus of example 14, wherein: the expected PCAD system pressure at the second time is defined as the sum of P1 and the product of (a) P1 on an absolute pressure scale (P1, ABS) over T1 on an absolute temperature scale (T1, ABS) and (b) the difference in T2 and T1, P1 is expressed on one of the absolute pressure scale and a complementary pressure scale, and T1 and T2 are expressed on the same temperature scale, said same temperature scale being one of the absolute temperature scale and a complementary temperature scale.

Example 16. The apparatus of any of examples 14-15, wherein the leak detection module is operative to convert P1 into P1, ABS and to convert T1 into T1, ABS.

Example 17. The apparatus of any of examples 14-16, wherein the leak detection module is operative to determine that there is a leak in the PCAD system when P2 is one of less than the expected PCAD system pressure and more than insubstantially less than the expected PCAD system pressure.

Example 18. The apparatus of any of examples 1-17, wherein the leak control module is operative to cease actuation of the PCAD system upon a determination that there is a leak in the PCAD system.

Example 19. A method comprising: determining there is a leak in a pneumatic cardiac assist device (PCAD) system over a period of time defined by a first time and a second time, wherein: the determination is based on a first PCAD system pressure at the first time (P1), a first PCAD system temperature at the first time (T1), a second PCAD system pressure at the second time (P2), and a second PCAD system temperature at the second time (T2) while accounting for a change in PCAD system pressure over the period of time attributable to a difference between T2 and T1, and P1, T1, P2, and T2 are associated with at least one component of the PCAD system; and controlling the PCAD system in response to the determination that there is a leak in the PCAD system.

Example 20. The method of example 19, wherein: the PCAD system includes a pneumatic drive unit, a driveline, and the PCAD, the pneumatic drive unit is operative to generate gas pressure and gas flow to operate the PCAD, the driveline is operative to shuttle the gas from the pneumatic drive unit to the PCAD, the PCAD is operative to provide therapeutic care to a patient, the PCAD is one of: an intra-aortic balloon, a patch device, and a cuff device, and the gas is air.

Example 21. The method of any of examples 19-20, wherein the leak detection module determines a leak using the Pressure-Temperature Gas Law.

Example 22. The method of any of examples 19-21, wherein accounting for the change in PCAD system pressure attributable to the difference between T1 and T2 comprises determining a temperature dependent pressure change at the second time based on the difference between T1 and T2, wherein the temperature dependent pressure change represents an expected difference in the PCAD system over the period of time given the change in T1 and T2.

Example 23. The method of example 22 wherein: the temperature dependent pressure change is defined as the product of (a) P1 on an absolute pressure scale (P1, ABS) over T1 on an absolute temperature scale (T1, ABS) and (b) the difference in T2 and T1, and T1 and T2 are expressed on the same temperature scale, said same temperature scale being one of the absolute temperature scale and a complementary temperature scale.

Example 24. The method of any of examples 22-23, further comprising determining a temperature compensated pressure at the second time based on P2 and the temperature dependent pressure change, wherein the temperature compensated pressure is defined as P2 reduced by the temperature dependent pressure change.

Example 25. The method of example 24, further comprising determining that the temperature compensated pressure is one of: less than P1 and more than insubstantially less than P1.

Example 26. The method of any of examples 19-25, wherein accounting for the change in PCAD system pressure attributable to the difference between T1 and T2 comprises determining an expected PCAD system pressure at the second time associated with the difference between T1 and T2.

Example 27. The method of example 26, wherein: the expected PCAD system pressure at the second time is defined as the sum of P1 and the product of (a) P1 on an absolute pressure scale (P1, ABS) over T1 on an absolute temperature scale (T1, ABS) and (b) the difference in T2 and T1, P1 is expressed on one of the absolute pressure sale and a complementary pressure scale, and T1 and T2 are expressed on the same temperature scale, said same temperature scale being one of the absolute temperature scale and a complementary temperature scale.

Example 28. The method of example 26, further comprising determining that P2 is one of: less than the expected PCAD system pressure and more than insubstantially less than the expected PCAD system pressure.

Example 29. The method of any of examples 19-28, further comprising ceasing actuation of the PCAD system.

Example 30. The apparatus of any of examples 1-5, wherein the apparatus includes the PCAD system and wherein: the PCAD system is configured to operate in both a closed mode and in an open mode, when the PCAD system is configured to operate in a closed mode, air is not permitted to enter or escape the system, when the PCAD system is configured to operate in an open mode, air is permitted to enter and escape the system; and each of the first time and the second time are times during which the PCAD system is operating in closed mode.

### General Considerations

As used herein, “module” may refer to any single or collection of circuit(s), integrated circuit(s), hardware processor(s), processing device(s), transistor(s), non-transitory memory(s), storage devices(s), non-transitory computer readable medium(s), combination logic circuit(s), or any combination of the above that is capable of providing a desired operation(s) or function(s). For example, “module” may take the form of a hardware processor executing instructions from one or more non-transitory memories, storage devices, or non-transitory computer readable media, or a dedicated integrated circuit. “Non-transitory memory,” “non-transitory computer-readable media,” and “storage device” may refer to any suitable internal or external non-transitory, volatile or non-volatile, memory device, memory chip(s), or storage device or chip(s) such as, but not limited to system memory, frame buffer memory, flash memory, random access memory (RAM), read only memory (ROM), a register, a latch, or any combination of the above. A “hardware processor” may refer to one or more dedicated or non-dedicated: hardware micro-processors, hardware micro-controllers, hardware sequencers, hardware micro-sequencers, digital signal hardware processors, hardware processing engines, hardware accelerators, applications specific circuits (ASICs), hardware state machines, programmable logic arrays, any integrated circuit(s), discreet circuit(s), etc. that is/are capable of processing data or information, or any suitable combination(s) thereof. A “processing device” may refer to any number of physical devices that is/are capable of processing (e.g., performing a variety of operations on) information (e.g., information in the form of binary data or carried/represented by any suitable media signal, etc.). For example, a processing device may be a hardware processor capable of executing executable instructions, a desktop computer, a laptop computer, a mobile device, a hand-held device, a server (e.g., a file server, a web server, a program server, or any other server), any other computer, etc. or any combination of the above. An example of a processing device may be a device that includes one or more integrated circuits comprising transistors that are programmed or configured to perform a particular task. “Executable instructions” may refer to software, firmware, programs, instructions or any other suitable instructions or commands capable of being processed by a suitable hardware processor.

While illustrative embodiments have been described herein, the scope thereof includes any and all embodiments having equivalent elements, modifications, omissions, combinations (e.g., of aspects across various embodiments), adaptations and/or alterations as would be appreciated by those in the art based on the present disclosure. For example, the number and

While various inventive embodiments have been described and illustrated herein, those of ordinary skill in the art will readily envision a variety of other means, steps, components, and/or structures for performing the function of the embodiments (and elements thereof) disclosed herein, and/or obtaining the results and/or one or more of the advantages described herein, and each of such variations and/or modifications is deemed to be within the scope of the inventive embodiments described herein. More generally, those skilled in the art will readily appreciate that all parameters, amounts, dimensions, materials, steps, and configurations described herein are meant to be merely an example and that the actual parameters, amounts, dimensions, materials, steps, and configurations will depend upon the specific application or applications for which the inventive teachings islare used. Those skilled in the art will recognize or be able to ascertain using no more than routine experimentation, many equivalents to the specific inventive embodiments described herein. It is therefore to be understood that the foregoing embodiments are presented by way of example only and that, within the scope of claims supported by the subject disclosure and equivalents thereto, and inventive embodiments may be practiced otherwise than as specifically described and claimed. Inventive embodiments of the present disclosure are directed to each individual feature, device, system, article, material, kit, step, function/functionality, and method described herein. In addition, any combination of two or more such features, devices, systems, articles, materials, kits, steps, functions/functionality, and methods, if such features, systems, articles, materials, kits, steps, functions/functionality, and methods are not mutually inconsistent, is included within the inventive scope of the present disclosure and considered embodiments.

Embodiments disclosed herein may also be combined with one or more features, components, materials, parameters, as well as complete systems, devices, and/or methods, to yield yet other embodiments and inventions. Moreover, some embodiments, may be distinguishable from the prior art by specifically lacking one and/or another feature disclosed in the particular prior art reference(s), i.e., claims to some embodiments may be distinguishable from the prior art by including one or more negative limitations.

Also, as noted, various inventive concepts may be embodied as one or more methods, of which one or more examples have been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments, orientation of components shown in the exemplary systems may be modified. Accordingly, those skilled in the art will appreciate that the method depicted in FIG. 7 may be altered in a variety of ways (e.g., the order of the acts may be rearranged; some acts may be performed in parallel shown acts may be omitted, or other acts may be included; a shown act may be divided into sub acts, or multiple shown acts may be combined into a single act, etc.).

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to) for a particular embodiment(s).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising.” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. An apparatus comprising:
a leak detection module operative to determine if there is a leak in a pneumatic cardiac assist device (PCAD) system over a period of time defined by a first time and a second time, wherein:
the determination is based on a first PCAD system pressure at the first time (P1), a first PCAD system temperature at the first time (T1), a second PCAD system pressure at the second time (P2), and a second PCAD system temperature at the second time (T2), while accounting for a change in PCAD system pressure over the period of time attributable to a difference between T2 and T1,
P1, T1, P2, and T2 are associated with at least one component of the PCAD system,
the leak detection module is configured to determine a leak using the Pressure-Temperature Gas Law,
the leak detection module is configured to determine a temperature dependent pressure change at the second time based on the difference between T1 and T2, the temperature dependent pressure change representative of an expected difference in the PCAD system over the period of time given the change in T1 and T2, and
the temperature dependent pressure change is defined as a product of (a) P1 on an absolute pressure scale (P1, ABS) over T1 on an absolute temperature scale (T1, ABS) and (b) a difference in T2 and T1, T1 and T2 are expressed on a same temperature scale, the same temperature scale being one of an absolute temperature scale and a complementary temperature scale; and
a leak control module that is operative to control the PCAD system in response to a determination that there is a leak in the PCAD system by the leak detection module.

2. The apparatus of claim 1, further comprising:
a pressure sensor operative to determine P1 and P2; and a temperature sensor operative to determine T1 and T2.

3. The apparatus of claim 2, wherein:
the PCAD system includes a pneumatic drive unit, a driveline, and the PCAD, the pneumatic drive unit is operative to generate gas pressure and gas flow to operate the PCAD,
the driveline is operative to shuttle the gas from the pneumatic drive unit to the PCAD, and
the PCAD is operative to provide therapeutic care to a patient.

4. The apparatus of claim 3, wherein the PCAD is one of: an intra-aortic balloon, a patch device, a cuff device, and a pump device.

5. The apparatus of claim 3, wherein the pneumatic drive unit comprises a manifold that is operative to communicate gas to the driveline, wherein:
P1 and P2 are the pressure of gas within the manifold at the first and second times, respectively, and
T1 and T2 are the temperature of gas within the manifold at the first and second times, respectively.

6. The apparatus of claim 3, wherein the gas is air.

7. The apparatus of claim 5, wherein the apparatus includes the PCAD system and wherein:
the PCAD system is configured to operate in both a closed mode and in an open mode,
when the PCAD system is configured to operate in a closed mode, air is not permitted to enter or escape the system,
when the PCAD system is configured to operate in an open mode, air is permitted to enter and escape the system; and
each of the first time and the second time are times during which the PCAD system is operating in closed mode.

8. The apparatus of claim 1, wherein the leak detection module is operative to convert P1 into P1, ABS and to convert T1 into T1, ABS.

9. The apparatus of claim 1, wherein the leak detection module is further operative to determine a temperature-compensated pressure at the second time based on P2 and the temperature dependent pressure change.

10. The apparatus of claim 9, wherein the temperature-compensated pressure is defined as P2 reduced by the temperature dependent pressure change.

11. The apparatus of claim 9, wherein the leak detection module is further operative to determine that there is a leak in the PCAD system when the temperature-compensated pressure is one of: less than P1 and more than insubstantially less than P1.

12. The apparatus of claim 1, wherein the leak detection module is operative to account for the change in PCAD system pressure attributable to the difference between T1 and T2 by determining an expected PCAD system pressure at the second time based on the difference between T1 and T2.

13. The apparatus of claim 12, wherein:
the expected PCAD system pressure at the second time is defined as the sum of P1 and the product of (a) P1 on an absolute pressure scale (P1, ABS) over T1 on an absolute temperature scale (T1, ABS) and (b) the difference in T2 and T1,
P1 is expressed on one of the absolute pressure scale and a complementary pressure scale, and
T1 and T2 are expressed on the same temperature scale, said same temperature scale being one of the absolute temperature scale and a complementary temperature scale.

14. The apparatus of claim 13, wherein the leak detection module is operative to convert P1 into P1, ABS and to convert T1 into T1, ABS.

15. The apparatus of claim 12, wherein the leak detection module is operative to determine that there is a leak in the PCAD system when P2 is one of: less than the expected PCAD system pressure and more than insubstantially less than the expected PCAD system pressure.

16. The apparatus of claim 1, wherein the leak control module is operative to cease actuation of the PCAD system upon a determination that there is a leak in the PCAD system.

17. A method comprising:
determining there is a leak in a pneumatic cardiac assist device (PCAD) system over a period of time defined by a first time and a second time, wherein:
the determination is based on the Pressure-Temperature Gas Law, a first PCAD system pressure at the first time (P1), a first PCAD system temperature at the first time (T1), a second PCAD system pressure at the second time (P2), and a second PCAD system temperature at the second time (T2) while accounting for a change in PCAD system pressure over the period of time attributable to a difference between T2 and T1,
P1, T1, P2, and T2 are associated with at least one component of the PCAD system,
wherein the accounting for the change in PCAD system pressure includes determining a temperature dependent pressure change at the second time based on the difference between T1 and T2, wherein the temperature dependent pressure change represents an expected difference in the PCAD system over the period of time given the change in T1 and T2, and
the temperature dependent pressure change is defined as the product of (a) P1 on an absolute pressure scale (P1, ABS) over T1 on an absolute temperature scale (T1, ABS) and (b) the difference in T2 and T1, and wherein T1 and T2 are expressed on the same temperature scale, the same temperature scale being one of the absolute temperature scale and a complementary temperature scale; and
controlling the PCAD system in response to the determination that there is a leak in the PCAD system.

18. The method of claim 17, wherein:
the PCAD system includes a pneumatic drive unit, a driveline, and the PCAD, the pneumatic drive unit is operative to generate gas pressure and gas flow to operate the PCAD,
the driveline is operative to shuttle the gas from the pneumatic drive unit to the PCAD,
the PCAD is operative to provide therapeutic care to a patient,
the PCAD is one of: an intra-aortic balloon, a patch device, and a cuff device, and the gas is air.

19. The method of claim 17, further comprising determining a temperature-compensated pressure at the second time based on P2 and the temperature dependent pressure change, wherein the temperature-compensated pressure is defined as P2 reduced by the temperature dependent pressure change.

20. The method of claim 19, further comprising determining that the temperature-compensated pressure is one of: less than P1 and more than insubstantially less than P1.

21. The method of claim 17, wherein accounting for the change in PCAD system pressure attributable to the difference between T1 and T2 comprises determining an expected PCAD system pressure at the second time associated with the difference between T1 and T2.

22. The method of claim 21, wherein:
the expected PCAD system pressure at the second time is defined as the sum of P1 and the product of (a) P1 on an absolute pressure scale (P1, ABS) over T1 on an absolute temperature scale (T1, ABS) and (b) the difference in T2 and T1,
P1 is expressed on one of the absolute pressure sale and a complementary pressure scale, and
T1 and T2 are expressed on the same temperature scale, said same temperature scale being one of the absolute temperature scale and a complementary temperature scale.

23. The method of claim 21, further comprising determining that P2 is one of: less than the expected PCAD system pressure and more than insubstantially less than the expected PCAD system pressure.

24. The method of claim 17, further comprising ceasing actuation of the PCAD system.
